(19) United States
(12) Patent Application Publication
(10) Pub. No.: US 2025/0009427 A1
(43) Pub. Date: Jan. 9, 2025

(54) SYSTEM AND METHOD FOR PIGMENT REMOVAL

(71) Applicant: EPILADY LLC, BROOKLYN, NY (US)

(72) Inventor: Yaniv YAKOV, Dover, DE (US)

(73) Assignee: EPILADY LLC, BROOKLYN, NY (US)

(21) Appl. No.: 18/711,508

(22) PCT Filed: Nov. 17, 2022
(86) PCT No.: PCT/US2022/050309

§ 371 Date: May 17, 2024

(30) Foreign Application Priority Data
Nov. 18, 2021 (EP) 21209111.0

(51) Int. Cl. A61B 18/20; A61B 18/00

(52) U.S. Cl. CPC A61B 18/203; A61B 2018/00452

(57) ABSTRACT

The present invention provides at least a system and a method, wherein the invention comprises at least one imaging component configured to extract target data and at least one radiation component. The system can further comprise a positioning component, the positioning component can be configured to position the radiation component based on the target data. In such embodiments the system can further comprise a data processing component, such as a CPU. The data processing component, imaging component and the radiation component can be configured to be installed in a handheld device.

### FIELD

The present invention is directed to a laser system and method to remove cutaneous pigmentation, preferably a handheld system.

### BACKGROUND

Cutaneous pigment removal has been performed with various tools, while often pigmentation, such as freckles, acne marks, lentigines, melasma, tattoo, age spots, post-inflammatory hyperpigmentation (PIH) are generally considered permanent, it is now possible to remove them, fully or partially. For example, the standard modality for tattoo removal is the non-invasive removal of tattoo pigments using Q-switched lasers. Different types of Q-switched lasers are used to target different colours of pigmentation depending on the specific light absorption spectra of the pigments. Typically, black and other darker-coloured sections can be removed completely using Q-switched lasers while lighter colours such as yellows and greens are still very difficult to remove. Success can depend on a wide variety of factors including skin colour, ink colour, and the depth at which the ink was applied.

With RU2692936C1, a device relating to medicine, namely to cosmetology and dermatology, can be used to remove tattoos on the skin. A fractional effect on the skin surface with a tattoo is carried out with a Nd: YAG Q-switch laser with an energy density of 5.5 J/cm², a laser flash generation frequency of 1 Hz with a laser beam spot diameter of 4-5 mm. The laser impact on the skin is carried out in a staggered manner in two stages: at the first stage, with the obligatory observance of the distance between the areas of exposure to the laser beam on the skin equal to the diameter of the treated areas, then at the second stage, after 48 hours, the laser is applied in the same mode to the tattoo areas not processed in the first session. Reprocessing of the tattoo is repeated no earlier than two months later. The method provides prevention of skin burns; reduction of tattoo removal time due to exposure of skin to a laser with high energy density on skin areas in a checkerboard pattern.

The Israeli company LIGHTSENSE LTD. has filed WO/2020/003138 and discloses methods and apparatus for dermatological laser treatment, e.g., for the removal of unwanted tattoos or other skin pigmentation. Removal of multiple colours with a single pulsed laser beam may be achieved using intensities in excess of about 50 GW/cm2. Methods for reducing the pain and tissue damage associated with laser tattoo removal include using a spot size of less than 2 mm with a fluence in the range 0.5-10 J/cm². Scanning the laser beam over an area of skin to be treated allows such areas to be treated accurately with scanning patterns calculated to promote rapid dissipation of heat away from treated portions of the skin. Multiple treatment rooms may be served by a single pulsed treatment laser by beam toggling, splitting or pulse-picking to minimize downtime of the laser.

DE102004006500 determines a dye colour to apply to the skin for tattooing or permanent skin make-up, whereby a skin examination area is illuminated with measurement light and the resultant reflected light analysed. A method for determining a colour value of an ink for tattooing or for application of permanent make up to the skin has the following steps: generation of light beams for illumination of an examination area of the skin; capture of measurement light reflected from the surface using detector that is sensitive over a number of spectral ranges and automatic processing of measurement light values using a processing unit to calculate a tattoo or permanent make up colour to apply to the skin. An independent claim is made for a device for determining a colour value of an ink for tattooing or permanent make-up of the skin.

US2007197883 discloses a spectroscopic diagnostic apparatus as an aid for laser tattoo Removal. A spectroscopic diagnostic apparatus is disclosed as an aid for laser tattoo removal. The apparatus performs spectroscopic analysis of the tattooed skin before or during laser treatment, which provides composition information of the tattoo pigments and photometric information of the skin for optimizing laser treatment protocols automatically or manually. It also provides a simulated treatment result for the selected laser types.

PCT/BR2008/000250 shows a surgical intradermal laser device for wrinkles, haemangiomas, hair follicles and tattoo removal through thermal stimulation or destruction of target-tissues with an optical fibre with spherical extremity which is used to conduct the laser energy to the subcutaneous tissue. The optical fibre goes through a needle, or catheter, which can be connected to a hand-piece device. The optical fibre goes completely through the layer of epidermis, and is introduced into any desired skin depth allowing the laser, to be applied directly on the target tissue.

ES2340566 relates to a procedure to remove pigmentary stains and tattoos on the skin, characterized in that comprises at least applying on the area to be treated a laser light emitted by a solid-state dye laser system, that tunes discrete wavelengths values comprised within the visible spectrum. Another object of the present invention is constituted by the same solid-state dye laser Irradiation system to remove pigmentary stains and tattoos on the skin according to the procedure described herein, as well as the active medium utilized in said system for generating and emitting laser light. Said active medium is characterized in that comprises at least one dye embedded in a solid matrix of at least one polymer, each dye-matrix combination emitting to a specific wavelength.

RU2550012C1 discloses sampling of biotic skin tissue with particles of implanted tattoo pigment. The samples are used to determine the tattoo pigment depth. The most effective laser wavelength is determined by exposing the tissue samples to laser light at various wave lengths. The samples are coloured, and those suffered the most severe damage of the tattoo pigment are detected. If the measured tattoo pigment depth is no more than 0.7 mm, the laser removal of the tattoo pigment is initiated. If the measured tattoo pigment depth falls within the range of 0.7 mm to 2.0 mm, the superficial destruction is expected to be followed by the laser removal of the tattoo pigment. The most effective laser wavelength determined by the biotic tissue sampled is specified for performing the removal procedure.

PCT/US1996/011384 discloses a laser treatment method which removes vascular and pigmented lesions from the skin of a living human. The methodology involves a carefully designed treatment protocol utilizing a modified optical apparatus. The apparatus is a modified diode laser system, designed for optimal therapeutic selectivity.

All existing methods and systems are dependent on the Fitzpatrick skin typing test. This means that the detection of a pigment depends on the colour of the skin of the recipient or the colour of the pigment. Additionally, the traditional methods and systems can be especially harmful when removing the tattoo pigments. Usually, the tattoo ink is targeted with some energy which causes the ink to break into segments. These ink segments are then found in the blood stream. The presence of these segments in the blood stream can lead to their accumulation in the lymph nodes, causing the enlargement of the lymph nodes and in some cases blood clots. The tattoo ink components also remain largely unknown and under-regulated, so there is a possibility of additional health risks.

### SUMMARY

In light of the above, it is an object of the present invention to overcome or at least alleviate the shortcomings of the prior art. More particularly, it is an object of the present invention to provide a system and a method for at least analysing cutaneous properties. It is a further object of the present invention to efficiently and painlessly remove cutaneous pigmentation.

These objects are met by the present invention. The present invention can be based on laser beams with wavelength in the near infrared (NIR) ranging between 500-1000 nm. The laser beams can comprise a width of 20 to 200 microns. These laser beams can then hit the epidermis layer of the skin. The epidermis layer can carry the pigmentation. The laser beam can penetrate the skin and warms up the pigment. This laser beam can further warm the surrounding skin along with the pigment. In some embodiments this can result in hundreds of micro tunnel depth being carved in the skin. This method can be effective in the revision of the skin. In some embodiments this can further be ejecting the pigment outside the body. In some embodiments this ejection can take up to 2-3 weeks of healing.

In some embodiments system can comprise a diode laser of 1 W, at most 3 W, which can be particularly advantageous for the ejection of the pigments from the skin. Further, the system can be configured to concentrate at least 2 laser beams to at least one target, in such embodiments the target can comprise one single area of the skin. This can cause the ejection of the pigment due to optical power per unit area of the skin.

In some embodiments the system can be configured to be fit in a smart home device, that can include taking photos of the skin. The home device can further be configured with a microscope. The home device can further be configured to analyse a target area using an image recognition algorithm. Further configured to automatically aim the laser beams to the target by a micro engines system.

Needless to describe that the pigments can comprise any sort of cutaneous pigment, natural or artificial. For example, tattoo ink, capillary veins, age spots, sun spots, acne spots and the likes. In some embodiments the target can comprise a skin area, in particular the skin area which can be covered by at least one exposure to the laser beam, in such embodiments the target size can be 80 mm² and it can take up to 3-5 seconds for at least one exposure.

One particular advantage of the present invention is that the system can be configured to deliver energy in the form of laser beams, the energy can be delivered in a manner where the energy is absorbed by the pigment and the surrounding skin tissues. In such embodiments the energy is the photo radiation energy. This can cause micro wounds in the skin.

In the following table various embodiments of the invention are shown:

| Wavelength | Fitzpatrick | Fitzpatrick | Fitzpatrick | Fitzpatrick | Fitzpatrick | Fitzpatrick |
|---|---|---|---|---|---|---|
| (nm) | 1 | 2 | 3 | 4 | 5 | 6 |
| 400-550 | ✓ | ✓ | | | | |
| 600-800 | ✓ | ✓ | ✓ | ✓ | | |
| 800-900 | | ✓ | ✓ | ✓ | ✓ | ✓ |
| 900-1000 | | | | | ✓ | ✓ |

To summarise, the above table:

In some embodiments the wavelength of the laser beam can be configured to be determined based on the Fitzpatrick skin type. For example, wavelength in the range of 400 to 550 nm can be used to remove pigmentation of the Fitzpatrick 1 skin type and/or Fitzpatrick 2 skin type.

In an embodiment a system comprises at least one imaging component configured to extract target data and at least one radiation component. The system can further comprise a positioning component, the positioning component can be configured to position the radiation component based on the target data. In such embodiments the system can further comprise a data processing component, such as a CPU. The data processing component, imaging component and the radiation component can be configured to be installed in a handheld device.

Further, the data processing component can comprise, a memory such as RAM. The data processing component can also comprise a storage. The storage can be local or on a remote server. In some embodiments the data processing component can be further configured to transfer the target data from the imaging component to the positioning component. In such embodiments the system can further comprise at least one of the existing communication protocols, to facilitate data exchange between the imaging component, positioning component and the data processing component.

In some embodiments the positioning component can comprise at least one of at least XY and at least XYZ positioning stage. This stage can be advantageous in providing a precise location to the positioning component. The target data extracted by the imaging component can further comprise a picture element, the picture element can further comprise pixels, defining the XYZ positioning of the positioning component. In some embodiments the data processing component can be configured to format the picture element in terms of XYZ positioning pixels. In some embodiments the picture element can comprise a picture element length can comprise a range of 1 to 100 mm, such that, 5 to 50 mm, preferably 10 to 20 mm. This range can comprise the size of the pigment or at least a portion of the pigment. Further, the picture element can comprise a picture element breadth which can be in the range of 1 to 100 mm, such that, 5 to 50 mm, preferably 10 to 20 mm.

In some embodiments the data processing component can be configured to determine a positioning range based on the parameters of the target data, wherein the parameters of the target data may comprise at least one of the at least the picture element length and the at least picture element breadth. The target data can further comprise a XY and/or XYZ coordinate and/or pixel data for the at least one target.

In some embodiments the radiation component can comprise at least one laser source. In some further embodiments the radiation component can comprise at least one plurality of laser sources. The at least one of laser source can further comprise a diode laser. The said laser source can comprise the diode laser, wherein the diode laser comprises power in range of 0.1 to 4 W, such as 0.5 to 2 W, preferably 0.8 W.

In some embodiments the radiation component can comprise at least one laser source, in such embodiments the laser source can further be configured with a laser beam comprising a width in range of 10 to 500 microns, such as 50 to 200 microns. Further, the at least one laser source can comprise a wavelength in near infrared range, such that 500 nm to 1000 nm. In some further embodiments the at least one laser source can comprise laser with wavelength in a visible range.

In some embodiments the radiation component can be configured with the laser source, wherein the laser source can further be configured with at least one array of laser beams. In such embodiments the system can be configured to position the laser source, such that the laser beam is delivered to a target. In such embodiments the positioning component can be configured to position the radiation component, with the XYZ/XY staging. The target in such embodiments can be located by the data processing component based on the target data. In some embodiments the target can be automatically determined by the data processing component.

In some embodiments the target can comprise a spot, in such embodiments the spot can comprises a diameter within a range of 0.01 mm to 1 mm, such that 0.1 mm. The spot can be a position on the skin and/or in the picture element. The positioning component can further be configured to position the laser beam based on the spot.

In some embodiments the radiation component can be configured to continuously or partially continuously deliver the laser beam to the target for a pre-determined exposure time. In such embodiments the data processing component can be configured to determine the exposure time, based on the target data. In such embodiments the pre-determined exposure time can comprise a time in range of 50 mS to 500 mS, such that 80 mS to 250 mS, preferably 100 mS.

The target data can further comprise at least the colour of the pigment. In some embodiments the target data can at least comprise colour of the skin. In some embodiments the laser source can comprise a laser beam intensity of at least 8 KW/cm². The said parameters can be advantageous to deliver radiations in a concentrated manner. In some embodiments the at least one laser beam and/or an array of laser beam can comprise a continuation laser beam.

In some embodiments the laser source can comprise a laser pulse fluence in skin depth in range of 100 to 1500 J/cm², such that 500 to 1200 J/cm² preferably 800 J/cm², in such embodiments the laser beam can comprise a spot size of at most 0.5 mm².

In some embodiments the fluence of each laser pulse and/or the spot size of the laser beam is/are can be configured to be determined by the data processing component, such that the fluence is at least 24 J/cm². In some further embodiments the data processing can further be configured to determine the sport size.

In some further embodiments the spot can be configured to be heated by the radiation component, such that the spot comprises a temperature, such as a momentary temperature within a range of 50 to 200 C.

In some embodiments the system can be configured to deliver the laser beam and/or the array of the laser beam to a pre-determined depth into the target. In such embodiments the photo radiation energy from the laser beam is configured to be absorbed by at least a portion of the target. The pre-determined depth can comprise at least 100 μm, such that the laser beam at least penetrates the epidermis without. The pre-determined depth can further comprise at most 1900 μm.

In some embodiments the radiation source can further be configured to create at least one ablative tunnel, preferably by raising the temperature of the target to at least 50° C. The said tunnels can be created by creating micro wounds in the skin by the heating caused by the photo radiation energy of the radiation component.

In some embodiments the laser source can be configured to create at least one plurality of ablative tunnels, preferably by raising the temperature of the target to at least 50° C. In some further embodiments the ablative tunnels are created in the skin by the heating, in such embodiments the pigment can be broken into at least one fragment. The radiation component can be configured to break the pigment into fragment, preferably by heating the pigment and/or by raising the temperature of the target to at least 50 C.

In some embodiments the pigment can be broken into fragment or the plurality of fragments, wherein the fragment comprises a diameter of at least 1 micron, such that 6 microns. This can be particularly advantageous because said size of fragments cannot be absorbed by the lymphatic system. In some embodiments the system can further be configured to eject the at least one fragment preferably via the ablative tunnel. The said ejection can be due to evaporation. In some embodiments the ejection can be manually. In some further embodiments the system can comprise a security component. The security component may be configured with at least one or a plurality of capacitive sensing device(s) and/or photoelectric sensing device(s) and/or electromagnetic induction sensing device(s). The security component may further be configured with at least one or a plurality of accelerometer(s) and/or gyroscope(s), compass(s). In some further embodiments the system can comprise a cooling component.

In a second embodiment a method is disclosed, wherein the method is configured to be performed on the system.

The present technology is also defined by the following numbered embodiments.

Below, system embodiments will be discussed. These embodiments are abbreviated by the letter “S” followed by a number. Whenever reference is herein made to “system embodiments”, these embodiments are meant.

S1. A system, comprising:
at least one imaging component configured to extract target data; and
at least one radiation component.

S2. The system according to the preceding embodiment wherein a positioning component is configured to position the radiation component based on the target data.

S3. The system according to the preceding embodiment wherein the system further comprises a data processing component.

S4. The system according to any of the preceding embodiments wherein the data processing component is configured to transfer the target data from the imaging component to the positioning component.

S5. The system according to the preceding embodiment wherein the positioning component comprises a XY and/or a XYZ positioning stage.

S6. The system according to any of the preceding embodiments wherein the target data comprises a picture element, wherein the picture element length comprises a range of 1 to 100 mm, such that, 5 to 50 mm, preferably 10 to 20 mm.

S7. The system according to any of the preceding embodiments wherein the target data comprises the picture element, wherein the picture element breadth comprises a range of 1 to 100 mm, such that, 5 to 50 mm, preferably 10 to 20 mm.

S8. The system according to any of the preceding embodiments wherein the data processing component is configured to determine a positioning range based on the parameters of the target data.

S9. The system according to any of the preceding embodiments wherein the target data comprises a XY and/or a XYZ coordinate data for the at least one target.

S10. The system according to any of the preceding embodiments wherein the radiation component comprises at least one laser source.

S11. The system according to any of the preceding embodiments wherein the radiation component comprises at least one plurality of laser sources.

S12. The system according to any of the preceding embodiments wherein the at least one laser source comprises a diode laser.

S13. The system according to any of the preceding embodiments wherein the at least one laser source comprises a diode laser, wherein the diode laser comprises power in range of 0.1 to 4 W, such as 0.5 to 2 W, preferably 0.8 W.

S14. The system according to any of the preceding embodiments wherein the radiation component comprises at least one laser source comprising a laser beam comprising a width in range of 10 to 500 microns, such as 50 to 200 microns.

S15. The system according to any of the preceding embodiments wherein the at least one laser source comprises a wavelength in a NIR (near infrared) range, such that 350 nm to 1000 nm.

S16. The system according to any of the preceding embodiments wherein the at least one laser source comprises a wavelength in a visible range.

S17. The system according to any of the preceding embodiments wherein the laser source comprises at least one array of laser beam.

S18. The system according to any of the preceding embodiments wherein the positioning component is further configured to position the laser source, such that the laser beam is delivered to a target.

S19. The system according to any of the preceding embodiments wherein the target is automatically determined by the data processing component, based on the target data.

S20. The system according to any of the preceding embodiments wherein the target comprises a spot, wherein the spot comprises a diameter within a range of 0.01 mm to 1 mm, such that 0.1 mm.

S21. The system according to any of the preceding embodiments wherein the radiation component is configured to continuously deliver the laser beam to the target for a pre-determined exposure time.

S22. The system according to the preceding embodiment wherein the pre-determined exposure time is configured to be determined by the data processing component, based on the target data.

S23. The system according to any of the preceding embodiments wherein the pre-determined exposure time comprises a time in range of 50 mS to 500 mS, such that 80 mS to 250 mS, preferably 100 mS.

S24. The system according to any of the preceding embodiments wherein the laser source comprises a laser beam intensity of at least 8 KW/cm².

S25. The system according to any of the preceding embodiments wherein the at least one laser beam and/or an array of laser beam comprises a continuation laser beam.

S26. The system according to any of the preceding embodiments wherein the laser source comprises a laser pulse fluence in skin depth in range of 100 to 1500 J/cm², such that 500 to 1200 J/cm² preferably 800 J/cm²

S27. The system according to any of the preceding embodiments wherein the laser beam comprises a spot size of at most 0.5 mm².

S28. The system according to any of the preceding embodiments wherein the fluence of each laser pulse and/or the spot size of the laser beam is/are configured to be determined by the data processing component, such that the fluence is at least 24 J/cm².

S29. The system according to any of the preceding embodiments and feature of S19 wherein the data processing component can be configured to determine the spot.

S30. The system according to any of the preceding embodiments wherein the radiation component is configured to heat the target, such that the spot comprises a temperature, such as a momentary temperature within a range of 50 to 200 C.

S31. The system according to any of the preceding embodiments wherein the system is further configured to deliver the laser beam and/or the array of the laser beam to a pre-determined depth into the target.

S32. The system according to any of the preceding embodiments wherein radiation from the laser beam is configured to be absorbed by the target.

S33. The system according to the preceding embodiment wherein the pre-determined depth comprises at least 100 μm.

S34. The system according to any of the preceding embodiments with the features of the preceding two embodiments wherein the pre-determined depth comprises at most 1900 μm.

S35. The system according to any of the preceding embodiments wherein the laser source is configured to create at least one ablative tunnel, preferably by raising the temperature of the target to at least 50° C.

S36. The system according to any of the preceding embodiments wherein the laser source is configured to create at least one plurality of ablative tunnels, preferably by raising the temperature of the target to at least 50° C.

S37. The system according to any of the preceding embodiments wherein the system is further configured to break the pigment into at least one fragment, preferably by raising the temperature of the target to at least 50 C.

S38. The system according to any of the preceding embodiments wherein the laser source is further configured to break the pigment into the at least one fragment.

S39. The system according to any of the preceding two embodiments wherein the at least one fragment comprises a diameter of at least 1 micron, such that 6 microns.

S40. The system according to any of the preceding embodiments wherein the at least one fragment comprises the diameter of at most 6 μm.

S41. The system according to any of the preceding embodiments wherein the system is further configured to eject the at least one fragment, preferably via the ablative tunnel.

S42. The system according to any of the preceding embodiments wherein the system comprises at least one security component.

S43. The system according to the preceding embodiment wherein the security component comprises at least one capacitance sensor, configured to sense the target.

S44. The system according to any of the preceding embodiments wherein the system comprises at least one cooling component.

Below, method embodiments will be discussed. These embodiments are abbreviated by the letter “M” followed by a number. Whenever reference is herein made to “method embodiments”, these embodiments are meant.

M1. A method comprising:
extracting target data;
providing at least one radiation component.

M2. The method according to the preceding embodiment wherein the method further comprises positioning the radiation component based on the target data using a positioning component.

M3. The method according to any of the preceding embodiments comprises providing a data processing component.

M4. The method according to any of the preceding embodiments wherein the data processing component comprises transferring the target data from the imaging component to the positioning component.

M5. The method according to any of the preceding embodiments wherein the target data comprises a picture element, wherein the picture element length comprises a range of 1 to 100 mm, such that, 5 to 50 mm, preferably 10 to 20 mm.

M6. The method according to any of the preceding embodiments wherein the target data comprises the picture element, wherein the picture element breadth comprises a range of 1 to 100 mm, such that, 5 to 50 mm, preferably 10 to 20 mm.

M7. The method according to any of the preceding embodiments wherein the data processing component comprises determining a positioning range based on the parameters of the target data.

M8. The method according to any of the preceding embodiments wherein the method comprises providing the target data with a XY and/or a XYZ coordinate data for the at least one target.

M9. The method according to any of the preceding embodiments wherein the method comprises providing the radiation component with at least one laser source.

M10. The method according to any of the preceding embodiments wherein the method comprises providing the radiation component with at least one plurality of laser sources.

M11. The method according to any of the preceding embodiments wherein the method comprises providing the at least one laser source with a diode laser.

M12. The method according to any of the preceding embodiments wherein the method comprises providing the at least one laser source with a diode laser, wherein the diode laser comprises as power in range of 0.1 to 4 W, such as 0.5 to 2 W, preferably 0.8 W.

M13. The method according to any of the preceding embodiments wherein the method comprises providing the radiation component with at least one laser source comprising a laser beam comprising a width in range of 10 to 500 microns, such as 20 to 200 microns.

M14. The method according to any of the preceding embodiments wherein the method comprises providing the at least one laser source with a wavelength in the NIR (near infrared) range and/or the visible range, such that 400 nm to 1000 nm.

M15. The method according to any of the preceding embodiments wherein the method comprises providing laser source with at least one array of laser beam.

M16. The method according to any of the preceding embodiments wherein the positioning component comprises positioning the laser source, such that the laser beam is delivered to a target.

M17. The method according to any of the preceding embodiments wherein the data processing component comprises automatically determining by the target, based on the target data.

M18. The method according to any of the preceding embodiments wherein the method comprises providing the target with a spot, wherein the spot comprises a diameter within a range of 0.01 mm to 1 mm, such that 0.1 mm.

M19. The method according to any of the preceding embodiments wherein the method comprises continuously delivering the laser beam to the target for a pre-determined exposure time.

M20. The method according to the preceding embodiment wherein the data processing component comprises determining the pre-determined exposure time, based on the target data.

M21. The method according to any of the preceding embodiments wherein the method comprises determining the pre-determined exposure time in range of 50 mS to 500 mS, such that 80 mS to 250 mS, preferably 100 mS.

M22. The method according to any of the preceding embodiments wherein the method comprises providing the laser source with a laser beam intensity of at least 8 KW/cm².

M23. The method according to any of the preceding embodiments wherein the method comprises providing at least one laser beam and/or an array of laser beam with a continuation laser beam.

M24. The method according to any of the preceding embodiments wherein the method comprises providing the laser source with a laser pulse fluence in skin depth in a range of 100 to 1500 J/cm², such that 500 to 1200, preferably 800 J/cm²

M25. The method according to any of the preceding embodiments wherein the method comprises providing the laser beam with a spot size of at most 0.5 mm².

M26. The method according to any of the preceding embodiments wherein the data processing component comprises determining the at least one of the fluence of each laser pulse and/or the spot size of the laser beam, such that the fluence is in a range of 100 to 1500 J/cm², such that 500 to 1200, preferably 800 J/cm².

M27. The method according to any of the preceding embodiments and feature of M18 wherein the data processing component comprises determining the spot size, such that the spot comprises a temperature within a range of 50 to 200 C.

M28. The method according to any of the preceding embodiments wherein the method further comprises delivering the laser beam and/or the array of the laser beam to a pre-determined depth into the target.

M29. The method according to the preceding embodiment wherein the method comprises providing the pre-determined depth with at least 100 μm.

M30. The method according to any of the preceding embodiments with the features of the preceding two embodiments wherein the pre-determined depth comprises at most 1900 μm.

M31. The method according to any of the preceding embodiments wherein the method comprises creating at least one ablative tunnel, preferably by raising the temperature of the target to at least 50° C.

M32. The method according to any of the preceding embodiments wherein the method further comprises creating at least one plurality of ablative tunnels, preferably by raising the temperature of the target to at least 50° C.

M33. The method according to any of the preceding embodiments wherein the method further comprises breaking a pigment into at least one fragment, preferably by raising the temperature of the target to at least 50° C.

M34. The method according to any of the preceding embodiments wherein the method further comprises breaking a pigment into at least one fragment using the radiation component.

M35. The method according to any of the preceding two embodiments wherein the method comprises providing at least one fragment with a diameter of at least 1 micron, such as 6 microns.

M36. The method according to any of the preceding embodiments wherein the at least one fragment comprises the diameter of at most 6 μm.

M37. The method according to any of the preceding embodiments wherein the method further comprises ejecting the at least one fragment, preferably via the ablative tunnel.

M38. The method according to any of the preceding embodiments wherein the method comprises providing at least one security component.

M39. The method according to the preceding embodiment wherein the method comprises providing the security component with at least one capacitance sensor, configured to sense the target.

M40. The method according to any of the preceding embodiments wherein the method comprises providing at least one cooling component.

Below, use embodiments will be discussed. These embodiments are abbreviated by the letter “U” followed by a number. Whenever reference is herein made to “use embodiments”, these embodiments are meant.

U1. Use of the system according to any of the preceding embodiments, for carrying out the method according to any of the preceding method embodiments.

U2. Use of the method according to any of the preceding method embodiments and the system according to any of the preceding embodiments, for removal of cutaneous pigmentation.

Below, program embodiments will be discussed. These embodiments are abbreviated by the letter “C” followed by a number. Whenever reference is herein made to “program embodiments”, these embodiments are meant.

C1. A computer-implemented program comprising instructions which, when executed by a user-device, causes the user-device to carry out the method steps according to any of the preceding method embodiments.

C2. A computer-implemented program comprising instructions which, when executed by a server, causes the at least one server to carry out the method steps according to any of the preceding method embodiments.

C3. A computer-implemented program comprising instructions which, when executed causes by a user-device, causes the user-device and a server to carry out the method steps according to any of the preceding method embodiments.

### BRIEF DESCRIPTION OF THE DRAWINGS

The present invention will now be described with reference to the accompanying drawings, which illustrate embodiments of the invention. These embodiments should only exemplify, but not limit, the present invention.

FIG. 1 depicts an embodiment of the present invention;

FIG. 2 depicts an embodiment of the present invention, wherein the present invention can be a handheld device;

FIG. 3 depicts an embodiment of the present invention;

FIG. 4 depicts an embodiment of the present invention;

FIG. 5 depicts an embodiment of the present invention;

FIG. 6 depicts an embodiment of an outcome of the use of the present invention;

FIG. 7 depicts an embodiment of an outcome of the use of the present invention.

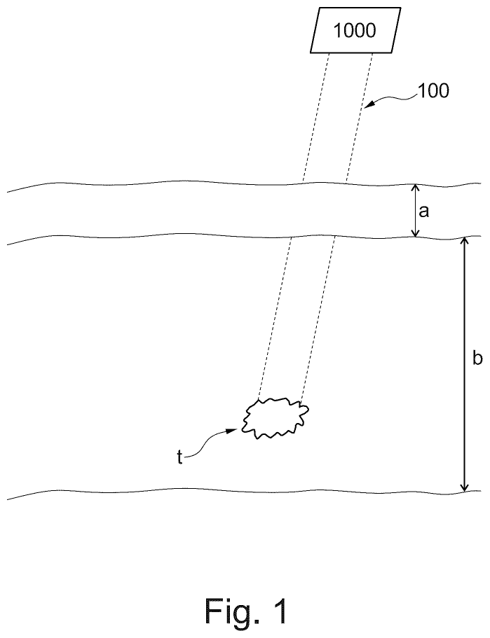

FIG. 1 shows the system 1000, configured with the radiation component, emitting energy, such as laser 100 on a pigment particle t stored in a target (dermis b). The laser 100 can be refracted when passing through the target (epidermis a). This refraction may be dependent on the absorption properties of the dermis a. The system 1000 can be configured with a data processing component, which can be also used to determine the optical properties of the target. The data processing component may comprise a computing unit. The computing unit can access the first data storage unit, the second data storage unit and the third data storage unit through the internal communication channel, which can comprise a bus connection. The at least one of the data storage units can comprise the target data. The at least one of the data storage units can comprise a knowledgebase.

The computing unit may be single processor or a plurality of processors, and may be, but not limited to, a CPU (central processing unit), GPU (graphical processing unit), DSP (digital signal processor), APU (accelerator processing unit), ASIC (application-specific integrated circuit), ASIP (application-specific instruction-set processor) or FPGA (field programmable gate array). The first data storage unit 30A may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

The second data storage unit may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

The third data storage unit may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

It should be understood that generally, the first data storage unit, the second data storage unit 30B, and the third data storage unit 30C can also be part of the same memory. That is, only one general data storage unit 30 per device may be provided, which may be configured to store the respective target data and the knowledgebase.

The data processing component may comprise a further memory component 140 which may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM). The memory component may also be connected with the other components of the data processing component (such as the computing component) through the internal communication channel.

Further the data processing component may comprise an external communication component. The external communication component may comprise an antenna (e.g., WIFI antenna, NFC antenna, 2G/3G/4G/5G antenna and the like), USB port/plug, LAN port/plug, contact pads offering electrical connectivity and the like. The external communication component can send and/or receive data based on a communication protocol which can comprise instructions for sending and/or receiving data

In addition, the data processing component may comprise an input user interface which can allow the user of the data processing component to provide at least one input (e.g., instruction) to the data processing component. For example, the input user interface may comprise a button, keyboard, trackpad, mouse, touchscreen, joystick and the like.

Additionally, still, the data processing component may comprise an output user interface which can allow the data processing component to provide indications to the user. For example, the output user interface may be a LED, a display, a speaker and the like.

The output and the input user interface may also be connected through the internal communication component with the internal component of the device. The data processing component may comprise a remote data processing component.

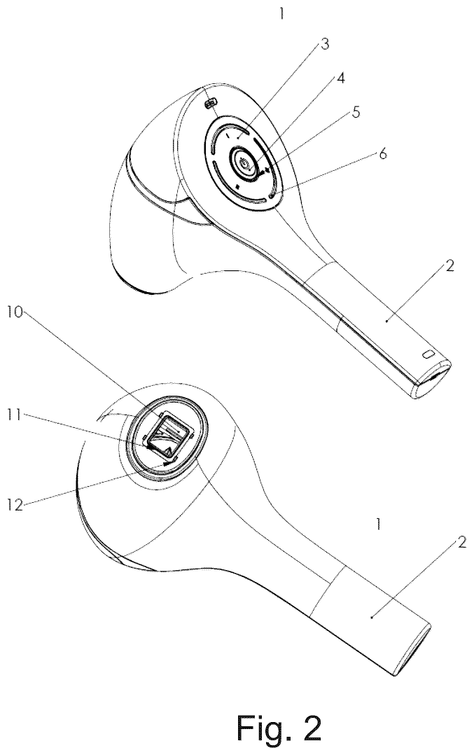

FIG. 2 is a schematic representation of the system 1, a handle 2, an operating panel 3 which can be configured with a power switch 4. The device can further comprise a and/or a plurality of battery indicators 6. The battery indicators 6 can be configured to display a visual indication of the battery's state of charge (SoC) or depth of discharge (DoD). The battery indicator 6 may be an LED battery level indicator, or an electronic display taking the form of a bar graph. The device may further comprise an aperture 11 and a targeting system 12 preferably at the perimeter of the aperture 11. The battery indicators 6 may also be indicating the power of the lasers. The device may further comprise a laser opening 10 which may be configured to allow radiations pass.

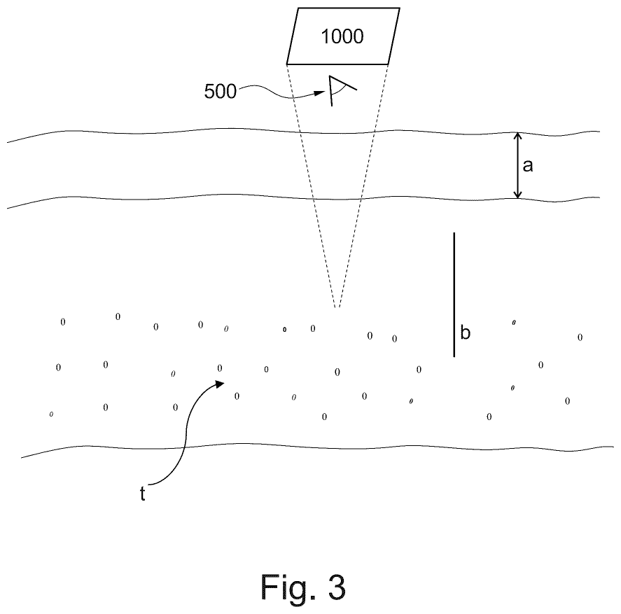

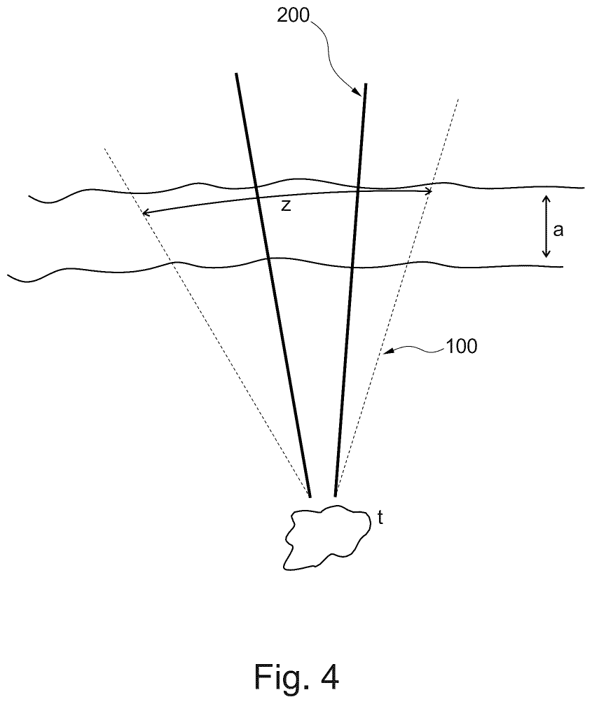

FIGS. 3 and 4 shows an embodiment of the present invention. The system 1000 can deliver the laser beam to the target t. The target t can be determined by the imaging component 500. The wavelength of the laser beam 100 from the system 1000 can be in the NIR at 500-1000 nm. The laser beams 100 can comprise a width of a few microns. The laser beam 100 can hit the epidermis layer b that covers the tattoo ink t, penetrates the skin and warms up the ink and the surrounding skin. As a result, hundreds of micros tunnel depth 200 can be carved in the skin. This procedure can be effective in the revision of the skin and ejecting the Tattoo ink outside the body after 2-3 weeks of healing. The invention can comprise a low power diode laser 100, for example up to 1 W, and can be used by concentrating 2-10 beams to one single small area spot, reaching a high number of optical powers to area unit. This fact can make it possible to implement the system 1000 in a small smart home device.

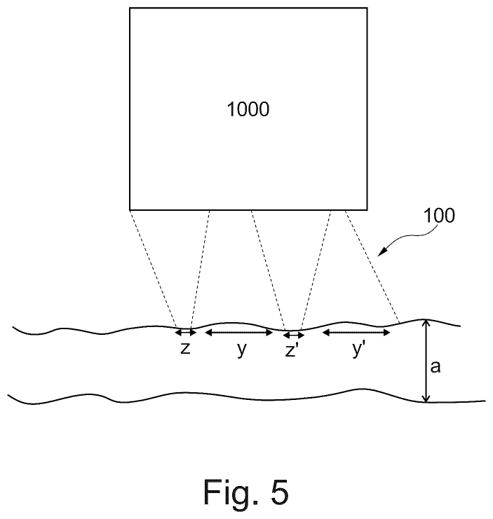

FIG. 5 shows an embodiment of the present invention wherein the spot size Z, Z′ of the laser beam is shown in comparison with the distance between the two spot sizes Y, Y′. As can be seen from the figure in some embodiments Z and Z′ are equal to Y and Y′. In some further embodiments they can be different.

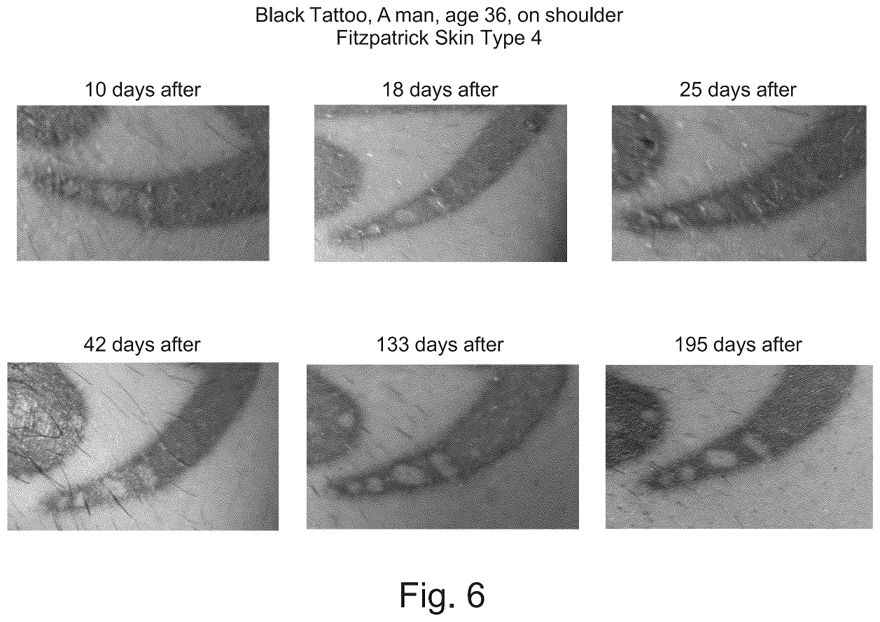

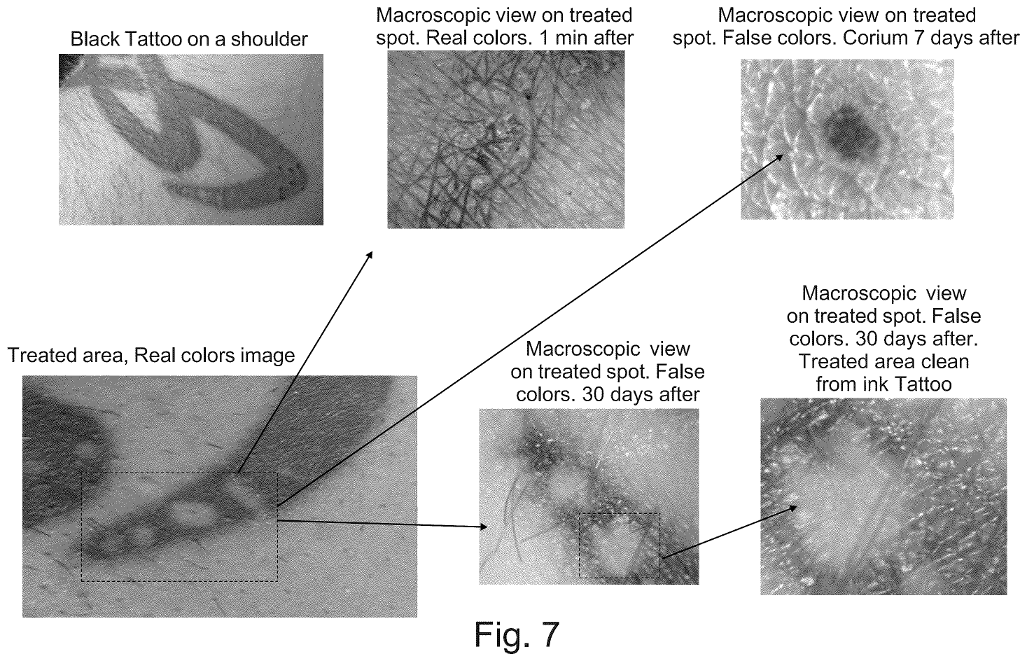

FIGS. 6 and 7 shows a use of the present invention. The system 1000 can be used for tattoo removal. In this embodiment a 36 years old man with skin type Fitzpatrick 4 can be seen. The black tattoo on the shoulder had not been treated before. The tattoo can be treated three times with three weeks intervals to get the shown result. The area where the laser 100 was delivered can be left uncovered. After the last follow up after 195 days, no hyper or hypopigmentation was observed. Normal skin regeneration was seen including normal hair growth.

In the following description, a series of features and/or steps are described. The skilled person will appreciate that unless explicitly required and/or unless requires by the context, the order of features and steps is not critical for the resulting configuration and its effect. Further, it will be apparent to the skilled person that irrespective of the order of features and steps, the presence or absence of time delay between steps can be present between some or all of the described steps.

It is noted that not all the drawings carry all the reference signs. Instead, in some of the drawings, some of the reference signs have been omitted for sake of brevity and simplicity of illustration. Embodiments of the present invention will now be described with reference to the accompanying drawings.

While in the above, a preferred embodiment has been described with reference to the accompanying drawings, the skilled person will understand that this embodiment was provided for illustrative purpose only and should by no means be construed to limit the scope of the present invention, which is defined by the claims.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of a first option and a second option” is intended to mean the first option or the second option or the first option and the second option.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

1. A system, comprising:
at least one imaging component configured to extract target data;
at least one radiation component; and
at least one positioning component configured to position the radiation component based on the target data.

2. The system according to claim 1, wherein the system further comprises a data processing component, wherein the data processing component is configured to transfer the target data from the imaging component to the positioning component.

3. The system according to claim 1, wherein the radiation component comprises at least one laser source, wherein the laser source comprises at least one diode laser.

4. The system according to claim 3, wherein the at least one diode laser comprises power in range of 0.1 W to 4 W.

5. The system according to claim 1, wherein the radiation component comprises the at least one laser source configured with a laser beam, wherein the laser beam width is in range of 10 microns to 500 microns.

6. The system according to claim 1, wherein the at least one laser source comprises radiations with wavelengths in range of 350 nm to 1000 nm.

7. The system according to claim 1, wherein the radiation component is configured to continuously deliver at least one laser beam to a target, wherein the intensity of the laser beam is at least 8 KW/cm².

8. The system according to claim 3, wherein the laser source comprises a laser pulse fluence in skin depth in range of 100 J/cm2 to 1500 J/cm².

9. The system according to claim 1, wherein the radiation component is configured to heat the target, such that the spot comprises a temperature, such as a momentary temperature within a range of 50° C. to 200° C.

10. The system according to claim 3, wherein the laser source is configured to create at least one ablative tunnel.

11. The system according to claim 1, wherein the system is further configured to break a pigment into at least one fragment, preferably by raising the temperature of the target to at least 50° C.

12. The system according to claim 11, wherein the at least one fragment comprises a diameter of at least 1 micron, such that 6 microns.

13. A method, comprising:
extracting target data, by at least one imaging component; and
providing at least one radiation component.

14. The method according to claim 13, comprises creating at least one ablative tunnel in a target.

15. The system according to claim 2, wherein the target data comprises at least one picture element.

16. The system according to claim 1, wherein the radiation component comprises the at least one laser source configured with a laser beam, wherein the laser beam width is in the range of 50 microns to 200 microns.

17. The system according to claim 3, wherein the laser source comprises a laser pulse fluence in skin depth in range of 500 J/cm2 to 1200 J/cm2.

18. The system according to claim 3, wherein the laser source comprises a laser pulse fluence in skin depth 800 J/cm².

19. The system according to claim 11, wherein the system is configured to eject the at least one fragment.

20. The method according to claim 14, wherein the at least one ablative tunnel is created by raising a temperature of the target to at least 50° C., further ejecting at least one fragment of a pigment via the ablative tunnel.
